(19) United States
(12) Patent Application Publication
(10) Pub. No.: US 2014/0303541 A1
(43) Pub. Date: Oct. 9, 2014

(54) Medical Devices, Wound Dressings, and Methods for Dressing Wounds

(71) Applicant: David J. Vachon, Spokane, WA (US)

(72) Inventor: David J. Vachon, Spokane, WA (US)

(21) Appl. No.: 13/299,196

(22) Filed: Nov. 17, 2011

Related U.S. Application Data
(60) Provisional application No. 61/414,598, filed on Nov. 17, 2010.
(60) Provisional application No. 61/449,038, filed on Mar. 3, 2011.

(51) Int. Cl. A61F 13/02; A61M 25/02

(52) U.S. Cl. 602/48; 604/180; 602/57; 602/54

(57) ABSTRACT

Medical devices, wound dressings, and methods of dressing wounds are described. Devices and methods using silicone and pharmaceutically active agents are described. Devices including covers and bases are described.

### CROSS REFERENCE TO RELATED APPLICATION

This application claims priority to U.S. Provisional Patent Application No. 61/414,598 which was filed on Nov. 17, 2010; U.S. Provisional Patent Application No. 61/449,038 which was filed on Mar. 3, 2011, the entirety of each of which is incorporated by reference herein.

### TECHNICAL FIELD

The disclosure relates generally to medical devices, barrier wound dressings, and methods for dressing wounds. More specifically, embodiments of the disclosure relate to surgical wound barrier dressings, including transcutaneous medical device, barrier dressings, antimicrobial adhesive barriers for surgical drapes, and antimicrobial adhesive barriers with absorbent surfaces, and processes for their production and use, for controlling infections and/or preventing and/or minimizing protein drug degradation, for a drug such as insulin, at the site of the delivery from an infusion set.

### BACKGROUND

There are various types of surgical procedures that create surgical wounds including investigative or corrective, minor or major, open (traditional) or minimal access surgery, elective or emergency, and/or incisions (simple cuts) or excision (removal of tissue). In each case surgery exposes sub-cutaneous tissue; the resulting wound requires management to promote satisfactory healing and to avoid complications such as infection. In the majority of cases this will require the use of some form of wound dressing. With ideal healing conditions, a surgical wound, whether an incision or excision, will follow the normal wound healing pathway associated with acute wounds. There are three accepted modes of healing. These include: Primary, Secondary, and tertiary intention healing. Primary intention healing involves bringing the edges of the wound together (in apposition) and securing them with sutures, clips or skin closure strips. These wounds usually seal within 24 to 48 hours and generally heal within 8 to 10 days. Healing takes place throughout the depth of the wound simultaneously and little new tissue has to be formed. There may be a little leakage from the wound for the first 2 days; the presence of bloody exudate or odor beyond this is a warning sign of a potential complication. Managing the risks associated with the open phase of healing is a prime consideration. Secondary intention healing requires that a wound is left open to allow healing by contraction and replacement of missing tissue with granulation and epithelial tissue. It is common for surgical excisions or traumatic wounds with tissue loss to be healed this way. The healing duration will depend on the amount of tissue that must be replaced and the resulting scar may be quite extensive. In tertiary intention healing, or delayed closure the wound is kept open to allow for drainage of exudate, control of contamination or for further surgical procedures to be completed. At a later date (usually within 7 days as bacterial contamination rises markedly from the 8th day onwards), the patient returns to the operating room for the wound to be surgically closed. If the wound has to be kept open for longer periods of time and there is significant bacterial contamination; this has to be reduced before the wound is closed.

Infection is of major concern to most clinicians, institutions and patients. Prior to the development of antiseptics (Lister ˜1860) and aseptic technique, infection rates following surgery were between 70% and 90% and 30% to 50% of these patients died as a consequence of these wound infections. Fortunately this situation has improved, with typical infection rates of ˜10% being quoted in clinical literature. However, even surgical units applying the most advanced and thorough aseptic protocols rarely produce infection rates below a 5% average. Surgical site infection (SSI) occurs in an estimated 15% of clean surgeries and 30% of contaminated surgeries. Plastic adhesive drapes with and without antimicrobial agents is a popular method of protecting the wound from SSI, but conflicting results place their efficacy in question. The embodiments disclosed herein can be used to improve the performance of surgical drapes.

The risk of infection is significantly higher in hospitals than in the home environment because the patient may be in a state of reduced immunity and may be exposed to micro-organisms against which an immune response has not been prepared. The development of an infection adds a substantial cost to treatment. A two-year, retrospective control study undertaken at the Alfred Hospital determined that the incremental cost attributable to surgical site infection after undergoing Coronary Artery Bypass Graft Surgery (CABG) was $12,419. The magnitude of the incremental cost was driven by an increase in the length of hospital stay and the required drug therapy. Thus, infection is potentially a considerable financial drain on valuable patient care resources.

The risk of surgical incision complication is increased in certain categories of patients including: the elderly or very young, immuno-compromised or immuno-suppressed, those with underlying debilitating disease, the nutritionally deprived, and those taking drugs and therapeutic treatments that reduce their ability to withstand infection.

The literature recognizes the criteria for an effective post-operative dressing as possessing high moisture vapor permeability (MVP), low adherence to the wound surface, absorbency, waterproof, or wash-proof for minor surgery, bacterial barrier, conformable, non-sensitizing, good adhesion to skin, sterile, low cost, non-flammable and non-toxic.

Transcutaneous medical devices are catheters, pins, implants and the like which pass through the skin, are indwelling for some considerable time, and reside inside and outside of the body during the course of function. Transcutaneous medical devices include but are not limited to central venous catheters, peripheral venous catheters, Swan-Gaus pulmonary catheters, central nervous system implants (ex. external ventricular drainage and ventricular reservoirs), peritoneal dialysis catheters, such as for continuous ambulatory peritoneal dialysis and continuous cyclic peritoneal dialysis, hemodialysis catheters, transvenous pacemaker leads and temporary orthopedic pins. Transcutaneous medical devices, when in place, have a portion of the device which is external, that is which is left protruding from the skin, and which can be the cause of infection. The risk of acquiring infections from transcutaneous devices is high. For instance, the risk of acquiring catheter-related bloodstream infection ranges from 0.9 to 8%. These nosocomial bloodstream infections cause a case fatality of more than 20%, and account for an increase of thousands of dollars in hospital costs per infection, or tens of thousands of dollars per survivor in ICU needing an extra week of hospital stay. As for peritoneal dialysis, a very experienced center today still has a peritonitis rate of one episode per 15 to 25 patient months. The major sources of bacteria in these device-related infections are from surrounding skin.

Long-term transcutaneous devices such as catheters, certain biosensors, and infusion devices require patients to endure extended skin breaches (wounds) that can be difficult to manage clinically, i.e. infection and inflammation. These complications not only shorten device lifespan, but also compromise patient health. In the case of short-term, transcutaneous glucose sensors, FDA has approved their usage for 3-7 days for patients with diabetes. Infection can be an issue with these short-term sensors, although generally the sensors are removed prior to the development of a full-blown infection. The ability to extend the in vivo lifespan of these sensors from days to weeks can lower the cost of such a therapy; however the longer these devices remain implanted, the risk of infection and hence adverse effects will increase. Adverse effects seen at sites of transcutaneous device implantation, including glucose sensors include infection, irritation, redness, itching and inflammation. Such adverse effects lead to shortened device lifespan in vivo and in addition they can discourage patient involvement in implantable sensors. Clearly, decreasing the risk of infections and inflammation at sensor implantation sites would likely not only increase sensor lifespan, but also decrease associated complications and adverse events.

To prevent infections associated with transcutaneous medical devices and surgical wounds, antiseptic preparation of insertion/incision sites, the initial application of topical anti-microbial solutions such as alcohol or iodine to the insertion sites is known. For transcutaneous devices, a further topical ointment after insertion of the device, such as an ointment containing neomycin, polymyxin and bacitracin, has been shown to prevent catheter/device colonization/infection, but it may increase the risk of fungal infections. For surgical incision sites, dressing the wound with an antimicrobial wound dressing such as a silver-based wound dressing is known. Ointments may be inconvenient depending upon the type of wound as a consequence of having to continually apply a dosing and the difficulty associated with being able to view the wound for assessment, i.e. requiring multiple replacements or having to clean the wound prior to evaluation. There have also been attempts to prevent infection at the site by the addition of an antimicrobial cuff to the catheters at or below the entry point, i.e. with an anti-microbial agent impregnated in the cuff. Efforts to coat the catheters with anti-microbial agents are known. However, these efforts have failed in clinical trials. Presently, the most common catheter dressing used in hospitals comprises sterile gauze or polyurethane film, both which have limited infection control properties. Efforts to replace gauze with a transparent film dressing to allow a visual check on the insertion site are known, see for instance U.S. Pat. No. 5,372,589, issued Dec. 13, 1994 to Davis. No anti-microbial control is taught with such a dressing. Johnson & Johnson Medical Inc. markets a product under the trademark BIOPATCH®, a chlorhexidine gluconate-impregnated polyurethane sponge catheter patch. An iodophor transparent dressing has also been suggested. However, to date, no completely effective anti-microbial device for use with transcutaneous medical devices is known. A layered antimicrobial device for securing transcutaneous devices is described by Burrell et al. in U.S. Pat. No. 7,137,968.

### BRIEF DESCRIPTION OF THE DRAWINGS

Embodiments of the disclosure are described below with reference to the following accompanying drawings.

FIG. 1 is a medical device according to an embodiment of the disclosure.

FIG. 2 is a medical device applied to a patient according to an embodiment of the disclosure.

FIG. 3 is a schematic sectional of a medical device such as a conformal surgical wound device barrier (island) dressing where the wound contact layer is fabricated from an absorbent material and the surrounding silicone gel barrier is formulated to include an antimicrobial agent, according to an embodiment of the disclosure.

FIGS. 4A and 4B are schematic sectionals of a medical device such as an antimicrobial adhesive “frame” barrier for placement around a surgical site. The barrier can be a three-layer construct comprising a top absorbent layer, a tacky silicone gel barrier formulated to include an antimicrobial agent, and a release liner that can be removed just prior to application, according to an embodiment of the disclosure.

FIG. 5 is a representation of a medical device such as a surgical barrier border placed on a patient's abdomen prior to a surgical procedure, according to an embodiment of the disclosure.

FIGS. 6A, 6B, and 6C are schematic sectionals of a medical device such as an island dressing including a flexible impervious layer and high surface area fabric composite beneath a tacky silicone gel layer with an absorbent “island” pad and release liner upper layer, according to an embodiment of the disclosure.

FIG. 7 is a schematic of a representative process flow that will allow for high volume production of a medical device in accordance with embodiments of the present disclosure.

FIG. 8 is a schematic sectional figure of a medical device such as a two-layer transcutaneous device dressing in accordance with embodiments of the present disclosure.

FIG. 9 is a schematic perspective view of a medical device such as a three-layer transcutaneous device composed of a cover and base mounted on opposite sides of a common substrate to form a top securing layer and a bottom dressing showing the opening for placement around the transcutaneous medical device, according to an embodiment of the disclosure.

FIG. 10 is a schematic perspective view of the device of FIG. 9 in another configuration, according to an embodiment of the disclosure.

FIG. 11 is a schematic cross sectional figure of a two-layer transcutaneous device dressing in place with a catheter penetrating the skin of a patient, according to an embodiment of the disclosure.

FIG. 12 is a schematic sectional of a two-component, two layer transcutaneous device dressing where the inner component is fabricated from a silicone gel formulated to contain a water absorbing material according to an embodiment of the disclosure.

FIG. 13 is a plan view of the transcutaneous device barrier dressing slid in place with a biosensor surrounded by a single dressing, such that the dressing is in contact with a portion of the sensor protruding from the skin according to an embodiment of the disclosure.

FIG. 14 is a plan view of the transcutaneous device dressing, according to an embodiment of the disclosure, showing the slit in the lower dressing.

FIG. 15 is a schematic perspective view of a three-layer transcutaneous device composed of a cover and base, with the lower skin contacting base including an inner insert of a water absorbing material and portions mounted on opposite sides of a common substrate to form a top securing layer and a bottom dressing showing the opening for placement around the transcutaneous medical device, according to an embodiment of the disclosure.

FIG. 16 is a chlorhexidine diacetate release profile (10% loading in silicone gel), according to an embodiment of the disclosure.

FIG. 17 is a polyhexanide hydrochloride release profile (10% loaded in silicone gel), according to an embodiment of the disclosure.

FIG. 18 is a polyhexanide hydrochloride release profile (10% loading in silicone gel with 10% polyvinylpyrrolidone blended into the silicone), according to an embodiment of the disclosure.

FIG. 19 is a chlorhexidine diacetate release profile (10% loading in silicone gel with 10% carboxymethylcellulose blended into the silicone), according to an embodiment of the disclosure.

FIG. 20 is a representation of the zones of clearance for 10, 15, & 20% chlorhexidine diacetate loaded Momentive Performance Materials UV-cured silicone gel, according to an embodiment of the disclosure.

FIG. 21 is a representation of the zones of clearance for 15% and 10% octenidine dihydrochloride loaded Nusil MED 6345 with A and B part ratios of 50:50, 45:55 respectively, and 10% octenidine loading in a 50:50 formulation and 0.5% and 10% polyhexanide hydrochloride with A and B part ratios of 50:50 each. FIG. 19 is a representation of the zones of clearance against Staphylococcus epidermidis (ATCC 12228) for 1.15% octenidine dihydrochloride (1:1, A:B), 2.10% octenidine dihydrochloride in Med 6345 (0.9:1.1, A:B), 3.10% octenidine dihydrochloride (1:1, A:B), 4. 0.5% PHMB (1:1, A:B), and 5.10% PHMB (1:1, A:B) all formulated in Nusil Med 6345. FIG. 20 is a representation of the zones of clearance against Staphylococcus aureus (clinical isolate) for 1.15% octenidine dihydrochloride (1:1, A:B), 2. 10% octenidine dihydrochloride in Med 6345 (0.9:1.1, A:B), 3.10% octenidine dihydrochloride (1:1, A:B), 4. 0.5% PHMB (1:1, A:B), and 5.10% PHMB (1:1, A:B) all formulated in Nusil Med 6345. FIG. 22 is a representation of the zones of clearance for 15% and 10% octenidine dihydrochloride loaded Nusil MED 6345 with A and B part ratios of 50:50, 45:55 respectively, and 10% octenidine loading in a 50:50 formulation and 0.5% and 10% polyhexanide hydrochloride with A and B part ratios of 50:50 each. FIG. 19 is a representation of the zones of clearance against Staphylococcus aureus (ATCC 12228) for 1.15% octenidine dihydrochloride (1:1, A:B), 2.10% octenidine dihydrochloride in Med 6345 (0.9:1.1, A:B), 3.10% octenidine dihydrochloride (1:1, A:B), 4. 0.5% PHMB (1:1, A:B), and 5.10% PHMB (1:1, A:B) all formulated in Nusil Med 6345. FIG. 20 is a representation of the zones of clearance against Staphylococcus aureus (clinical isolate) for 1.15% octenidine dihydrochloride (1:1, A:B), 2.10% octenidine dihydrochloride in Med 6345 (0.9:1.1, A:B), 3.10% octenidine dihydrochloride (1:1, A:B), 4. 0.5% PHMB (1:1, A:B), and 5.10% PHMB (1:1, A:B) all formulated in Nusil Med 6345.

### DESCRIPTION

This disclosure is submitted in furtherance of the constitutional purposes of the U.S. Patent Laws “to promote the progress of science and useful arts” (Article 1, Section 8).

Applicant has recognized numerous shortcomings of current securing devices. For example, a securing device is taught for affixing an intravenous device to the body in U.S. Pat. No. 3,918,446, issued Nov. 11, 1975 to Buttaravoli. The device has an upper and a lower pad, between which the intravenous device is fixed. Since the function of the device is to secure the device to the body, there is a teaching to provide an adhesive material to the bottom of lower pad, and to the bottom of the top pad. There is a mention of providing the adhesive with an antibacterial agent.

This device has the disadvantage of using adhesives with the antibacterial agent, which generally limits the effectiveness and long lasting ability of the antibacterial agent. Furthermore, the adhesive can be irritating next to the skin, cause skin damage and patient discomfort on removal, and inhibits the removal or changing of the device. Furthermore, many adhesives that are placed onto the surface of a matrix aimed at the delivery of an active pharmaceutical ingredient (API) can act as a moisture barrier, which can limit the effectiveness of the API because moisture generally facilitates the delivery of the API by first dissolving the API and subsequently aiding the release of the API from the material which has been placed at the site of interest, i.e. the site of transcutaneous device implantation. The term agent and ingredient may be used interchangeably herein.

The disclosed dressing is a waterproof, conformable dressing formulated with or without any variety of active pharmaceutical ingredients (such as an antimicrobial agent, antiproteolytic agent, or oligodynamic metal, and/or mixtures of thereof), the bulk dressing material possessing appreciable tack (and as such does not require the use of a secondary adhesive layer, although one can be used if desired) that will not damage epithelium upon removal, is easily constructed with or without an absorbent pad to collect wound exudate, and creates a seal (barrier) around the entire wound to limit any infiltration of invading pathogens to the incision site.

Embodiments of the device of the present disclosure can provide a skin friendly, conformal, tacky & self adhesive silicone gel construct compounded with an antimicrobial agent. The device can also include a protective top layer that acts as an adhesion layer in some embodiments, a peel away release liner, and a slit through the dressing exposing access to a central cutout that allows placement of the barrier dressing on the skin and below the transcutaneous device when in place thus allowing the transcutaneous device to remain in contact with the skin to provide a conformal antimicrobial barrier surrounding the device and therefore limiting the likelihood of infection of the device or the tissue surrounding the device. In one embodiment, the transcutaneous device dressing barrier is secured around the device and a second similar barrier device mounted on the opposite side of the protective barrier is secured over the top of the protruding transcutaneous device (such as a catheter) and to the top (protective) layer. In the case of a surgical wound dressing barrier device, a skin friendly, conformal, tacky & self adhesive silicone gel construct compounded with an antimicrobial agent. The dressing also includes a protective top layer that acts as an adhesion layer in some embodiments, a peel away release liner, and an absorbent pad in the center of the dressing (island) allows placement of the barrier dressing on the skin surrounding the surgical wound in order to provide a conformal antimicrobial barrier surrounding the wound and therefore limiting the likelihood of infection of the wound. In one embodiment, the surgical wound dressing barrier is secured around the wound and the absorbent material is in direct contact with the closed or open surgical wound, abrasion, or penetrating injury. In the case of a dry wound, a tacky silicone wound dressing barrier without an absorbent “island” can be utilized in order to protect the wound from external insult and invading pathogens.

Barrier wound dressings of the disclosure can include a first tacky silicone gel polymer interfacial adhesion layer formulated to include at least one active pharmaceutical agent are provided.

Barrier wound dressings and antimicrobial barrier adhesives including a first tacky silicone gel polymer interfacial adhesion layer to include an active pharmaceutical agent wherein said active pharmaceutical agent can include an antibacterial agent, anti-inflammatory agent, nutrient, antibiotic agent, healing agent, antiproteolytic agent, anesthetic agent, oligodynamic metal, are provided.

Tacky silicone gel polymer interfacial adhesion layers incorporating an active pharmaceutical agent of organic, organometallic, or metallic nature comprising one or more of an antimicrobial agent, antibiotic agent, anti-inflammatory agent, antiproteolytic agent, anesthetic agent, nutritional agent, healing agent, coagulation agent, anticoagulation agent, oligodynamic metal, moisturizing agent, or an angiogenesis stimulating agent are provided.

Barrier wound dressings including tacky silicone gel polymer interfacial adhesion layers formulated to include at least one active pharmaceutical agent are provided.

Barrier wound dressings including a first tacky silicone gel polymer interfacial adhesion layer including an active pharmaceutical agent wherein said active pharmaceutical agent can include one or more of an antibacterial agent, anti-inflammatory agent, nutrient, antibiotic agent, healing agent, or anesthetic agent are provided.

Single dressing barrier wound dressings including a single tacky silicone gel polymer interfacial adhesion layer adapted for application upon a tissue surface and surrounding the medical device at the location where the device breaches the body surface modified to include a central inner absorbent material component formulated to include at least one active pharmaceutical agent are provided.

Also provided are barrier wound dressings including a single tacky silicone gel polymer interfacial adhesion layer adapted for application upon a tissue surface and encompassing a surgically created incision site whereby a non-adherent, fluid-absorbing layer is in contact with the closed, or open, incision, and the conformal “tacky” silicone interfacial adhesion layer is adhered to skin surrounding the surgical wound. The silicone interfacial adhesion layer may be formulated to include at least one active pharmaceutical agent or one of many non-active compounds that may aid in the healing of the surgical wound. It is understood that dressings created for the purposes of dressing surgical wounds can be used to dress wounds resulting from injury or trauma.

Surgical site barriers including a single tacky silicone gel polymer interfacial layer comprising a top (upper) layer of high surface area material for the absorbance of fluids are provided in roll format and the conformal “tacky” silicone layer can be adhered to the skin surrounding the site where the surgery may be conducted. The silicone interfacial adhesion layer may be formulated to include at least one active pharmaceutical agent or one of many non-active compounds that may aid in preventing bacterial infiltration of the surgical wound.

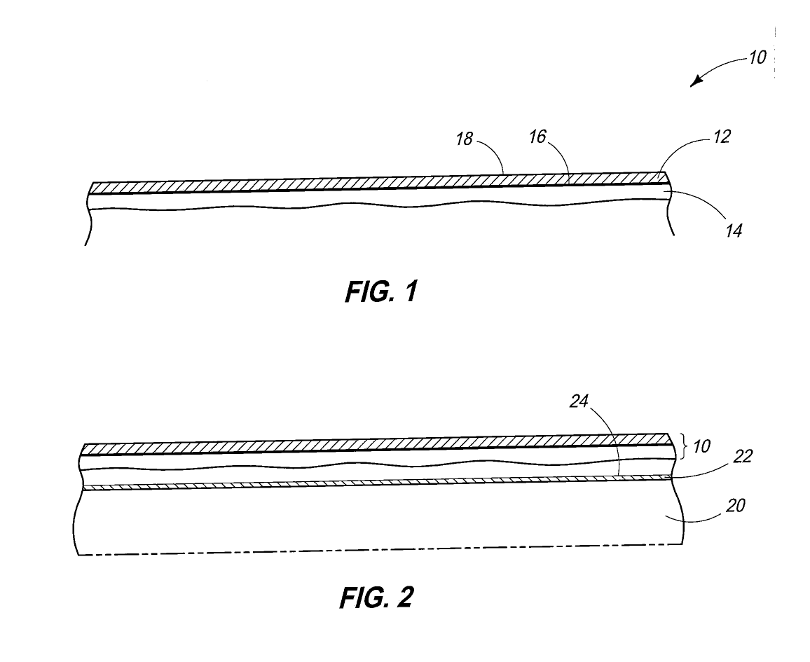

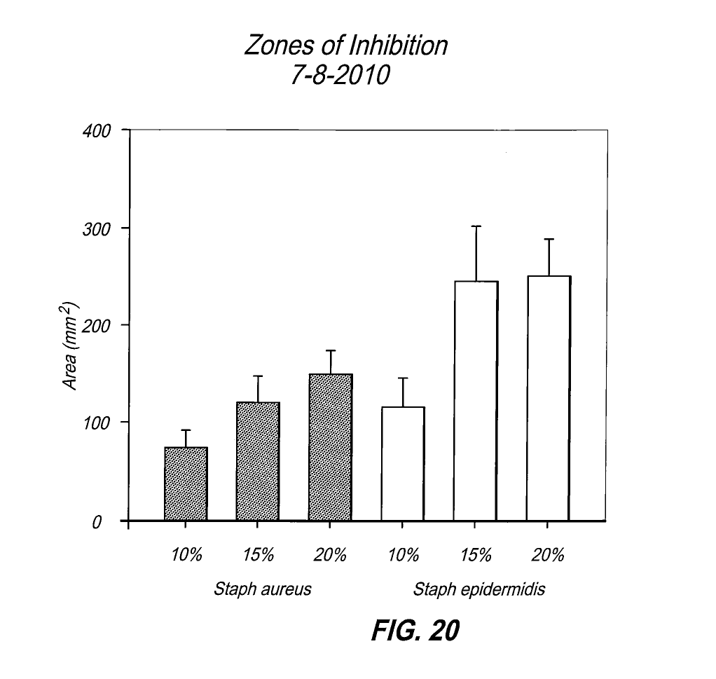

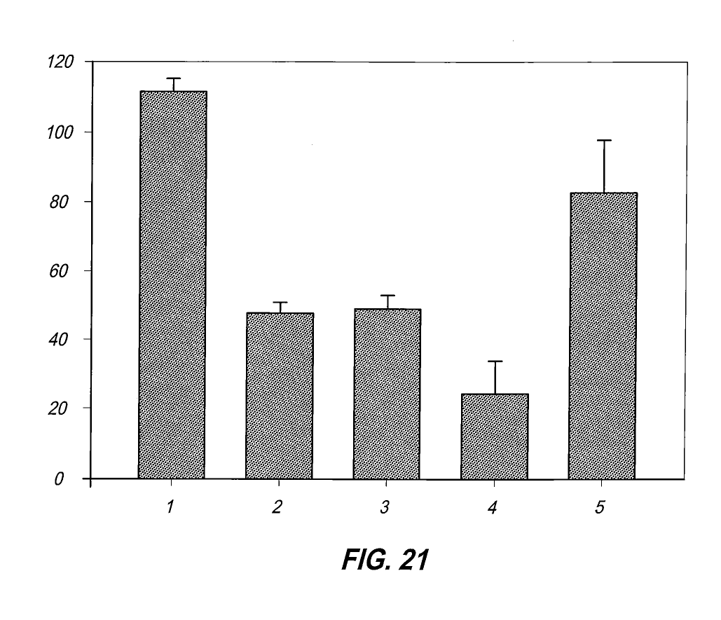

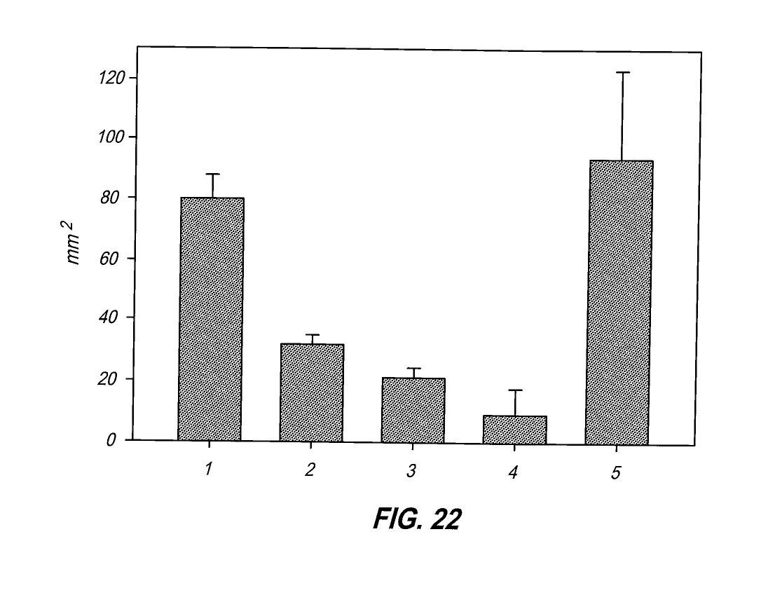

The devices and methods of the present disclosure are described with references to FIGS. 1-22. Referring first to FIG. 1, a medical device 10 is shown that includes a substrate 12 having one surface 16 and another opposing surface 18. On the one surface 16 is a composition 14 comprising a silicone material and at least one pharmaceutically active agent.

In accordance with example implementations, the silicone material can be a tacky silicone material. Silicone is a polymer comprising siloxane units such as the dimethylsiloxane variety. The silicone material can be a gel having “tack” consistent with the amount of cross linking units present in the material. Generally, medical grade silicones are cured by one of three mechanisms. These include: 1. Room temperature vulcanization (RTV) which is either a single component silicone rubber precursor that can utilized condensation-type chemistry involving the loss of acetic acid during cure or as platinum catalyst-based addition chemistry; 2. Peroxide catalyzed vulcanization whereby two components are combined (one containing the catalyst) where the catalyst is a peroxide, such as benzoyl peroxide, which initiates a free radical reaction for vulcanization/crosslinking of the two components with the addition of heat; and 3. Platinum complex-mediated (addition cure) rubber that requires compounding of two components and heating or irradiating the rubber mixture to drive the reaction to completion.

The pharmaceutically active agent can be one or more of an antibacterial agent, anti-inflammatory agent, nutrient, antibiotic agent, healing agent, antiproteolytic agent, anesthetic agent, oligodynamic metal agent, coagulation agent, anticoagulation agent, moisturizing agent, and/or angiogenesis stimulating agent.

Antimicrobial agent can be a term for drugs, chemicals, or other substances that either kill or slow the growth of microbes. Among the antimicrobial agents are antibacterial drugs, including antibiotics, antiviral agents, antifungal agents, organometallic compounds such as silver carbenes, anti parasitic drugs, and oligodynamic metals to include silver and the like. Antibiotics are commonly classified based on their mechanism of action, chemical structure, or spectrum of activity. Most antibiotics target bacterial functions or growth processes. Antibiotics that target the bacterial cell wall (penicillins, cephalosporins), or cell membrane (polymoxins), or interfere with essential bacterial enzymes (quinolones, sulfonamides) are usually bactericidal in nature. Those that target protein synthesis, such as the aminoglycosides, macrolides, and tetracyclines, are usually bacteriostatic. Further categorization is based on their target specificity: “Narrow-spectrum” antibiotics target particular types of bacteria, such as Gram-negative or Gram-positive bacteria, whereas broad-spectrum antibiotics affect a wide range of bacteria. In the last few years, three new classes of antibiotics have been brought into clinical use. This follows a 40-year hiatus in discovering new classes of antibiotic compounds. These new antibiotics are of the following three classes: cyclic lipopeptides (daptomycin), glycyclines (tigecycline), and oxazolidinones (linezolid). Tigecycline is a broad-spectrum antibiotic, whereas the two others are used for Gram-positive infections. These developments show promise as a means to counteract the bacterial resistance to existing antibiotics.

The antimicrobial agent can include an “Anti-microbial metal” which can be metals whose ions have an anti-microbial effect. “Metal” or “metals” includes one or more metals whether in the form of substantially pure metals, alloys or compounds including oxides, and salts such as nitrides, borides, sulphides, halides, carboxylates, or hydrides. The metal may also be biocompatible. Anti-microbial metals include Ag, Au, Pt, Pd, Ir, Ga (i.e. the noble metals), Sn, Cu, Sb, Bi, Ce, and Zn. Atoms, ions, molecules or clusters of the anti-microbial metal (herein after “species” of the anti-microbial metal) can have “Anti-microbial effect” when they are released.

The antimicrobial agent can be one or more of chloroxylenol (parachlorometaxylenol), acedapsone; acetosulfone sodium; alamecin; alexidine; amdinocillin; amdinocillin; pivoxil; amicycline; amifloxacin; amifloxacinmesylate; amikacin; amikacin sulfate; aminosalicylic acid; aminosalicylate sodium; amoxicillin; amphomycin; ampicillin; ampicillin sodium; apalcillin sodium; apramycin; aspartocin; astromicin sulfate; avilamycin; avoparcin; azithromycin; azlocillin; azlocillin sodium; bacampicillin hydrochloride; bacitracin; bacitracin, methylenedisalicylate; bacitracin zinc; bambermycins; benzoylpas calcium; berythromycin; betamicin sulfate; biapenem; biniramycin; biphenamine hydrochloride; bispyrithionemagsulfex; butikacin; butirosin sulfate; capreomycin sulfate; carbadox; carbenicillin disodium; carbenicillin, indanyl sodium; carbenicillin phenyl sodium; carbenicillin potassium; carumonam sodium; cefaclor; cefadroxil; cefamandole; cefamandolenafate; cefamandole sodium; cefaparole; cefatrizine; cefazaflur sodium; cefazolin; cefazolin sodium; cefbuperazone; cefdinir; cefepime; cefepime hydrochloride; cefetecol; cefixime; cefinenoxime hydrochloride; cefinetazole; cefinetazole sodium; cefonicid monosodium; cefonicid sodium; cefoperazone sodium; ceforanide; cefotaxime sodium; cefotetan; cefotetan disodium; cefotiam hydrochloride; cefoxitin; cefoxitin sodium; cefpimizole; cefpimizole sodium; cefpiramide; cefpiramide sodium; cefpirome sulfate; cefpodoxime; proxetil; cefprozil; cefroxadine; cefsulodin sodium; ceftazidime; ceftibuten; ceftizoxime sodium; ceftriaxone sodium; cefuroxime; cefuroximeaxetil; cefuroximepivoxetil; cefuroxime sodium; cephacetrile sodium; cephalexin; cephalexin hydrochloride; cephaloglycin; cephaloridine; cephalothin sodium; cephapirin sodium; cephradine; cetocycline hydrochloride; cetophenicol; chloramphenicol; chloramphenicolpalmitate; chloramphenicolpantothenate complex; chloramphenicol sodium succinate; chlorhexidinephosphanilate; chlorhexidinediacetate, chlorhexidinedihydrochloride, chlorhexidinedigluconate, chlortetracycline bisulfate; chlortetracycline hydrochloride; cinoxacin; ciprofloxacin; ciprofloxacin hydrochloride; cirolemycin; clarithromycin; clinafloxacin hydrochloride; clindamycin; clindamycin hydrochloride; clindamycinpalmitate hydrochloride; clindamycin phosphate; clofazimine; cloxacillinbenzathine; cloxacillin sodium; cloxyquin; colistimethate sodium; colistin sulfate; coumermycin; coumermycin sodium; cyclacillin; cycloserine; dalfopristin; dapsone; daptomycin; demeclocycline; demeclocycline hydrochloride; demecycline; denofungin; diaveridine; dicloxacillin; dicloxacillin sodium; dihydrostreptomycin sulfate; dipyrithione; dirithromycin; doxycycline; doxycycline calcium; doxycyclinefosfatex; doxycyclinehyclate; droxacin sodium; enoxacin; epicillin; epitetracycline hydrochloride; erythromycin; erythromycin acistrate; erythromycin estolate; erythromycin ethylsuccinate; erythromycin gluceptate; erythromycin lactobionate; erythromycin propionate; erythromycin stearate; ethambutol hydrochloride; ethionamide; fleroxacin; floxacillin; fludalanine; flumequine; fosfomycin; fosfomycintromethamine; fumoxicillin; furazolium chloride; furazoliumtartrate; fusidate sodium; fusidic acid; ganciclovir and ganciclovir sodium; gentamicin sulfate; gloximonam; gramicidin; haloprogin; hetacillin; hetacillin potassium; hexedine; ibafloxacin; imipenem; isoconazole; isepamicin; isoniazid; josamycin; kanamycin sulfate; kitasamycin; levofuraltadone; levopropylcillin potassium; lexithromycin; lincomycin; lincomycin hydrochloride; lomefloxacin; lomefloxacin hydrochloride; lomefloxacinmesylate; loracarbef; mafenide; meclocycline; meclocyclinesulfosalicylate; megalomicin potassium phosphate; mequidox; meropenem; methacycline; methacycline hydrochloride; methenamine; methenamine hippurate; methenamine mandelate; methicillin sodium; metioprim; metronidazole hydrochloride; metronidazole phosphate; mezlocillin; mezlocillin sodium; minocycline; minocycline hydrochloride; mirincamycin hydrochloride; monensin; monensinsodiumr; monovalent silver salts, nafcillin sodium; nalidixate sodium; nalidixic acid; natainycin; nebramycin; neomycin palmitate; neomycin sulfate; neomycin undecylenate; netilmicin sulfate; neutramycin; nifuiradene; nifuraldezone; nifuratel; nifuratrone; nifurdazil; nifurimide; nifiupirinol; nifurquinazol; nifurthiazole; nitrocycline; nitrofurantoin; nitromide; norfloxacin; novobiocin sodium; octenidinedihydrochloride, octenidinediacetate, octenidinedigluconate, ofloxacin; onnetoprim; oxacillin and oxacillin sodium; oximonam; oximonam sodium; oxolinic acid; oxytetracycline; oxytetracycline calcium; oxytetracycline hydrochloride; paldimycin; parachlorophenol; paulomycin; pefloxacin; pefloxacinmesylate; penamecillin; penicillins such as penicillin g benzathine, penicillin g potassium, penicillin g procaine, penicillin g sodium, penicillin v, penicillin v benzathine, penicillin v hydrabamine, and penicillin v potassium; pentizidone sodium; phenyl aminosalicylate; piperacillin sodium; pirbenicillin sodium; piridicillin sodium; pirlimycin hydrochloride; pivampicillin hydrochloride; pivampicillinpamoate; pivampicillinprobenate; polyhexamethylenebiguanide (polyhexanide hydrochloride, PHMB); polymyxin b sulfate; porfiromycin; propikacin; pyrazinamide; pyrithione zinc; quindecamine acetate; quinupristin; racephenicol; ramoplanin; ranimycin; relomycin; repromicin; rifabutin; rifametane; rifamexil; rifamide; rifampin; rifapentine; rifaximin; rolitetracycline; rolitetracycline nitrate; rosaramicin; rosaramicin butyrate; rosaramicin propionate; rosaramicin sodium phosphate; rosaramicinstearate; rosoxacin; roxarsone; roxithromycin; sancycline; sanfetrinem sodium; sarmoxicillin; sarpicillin; scopafungin; silver acetate; silver nitrate, nanocrystalline silver, silver polystyrene sulfonate (cross-linked” and non-cross-linked); silver carboxymethyl cellulose, silver polysaccharides (such as silver chondroitin sulfate and the like), silver carbene compounds, sisomicin; sisomicin sulfate; sparfloxacin; spectinomycin hydrochloride; spiramycin; stallimycin hydrochloride; steffimycin; streptomycin sulfate; streptonicozid; sulfabenz; sulfabenzamide; sulfacetamide; sulfacetamide sodium; sulfacytine; sulfadiazine; sulfadiazine sodium; sulfadiazine silver; sulfadoxine; sulfalene; sulfamerazine; sulfameter; sulfamethazine; sulfamethizole; sulfamethoxazole; sulfamonomethoxine; sulfamoxole; sulfanilate zinc; sulfanitran; sulfasalazine; sulfasomizole; sulfathiazole; sulfazamet; sulfisoxazole; sulfisoxazole acetyl; sulfisboxazolediolamine; sulfomyxin; sulopenem; sultamricillin; suncillin sodium; talampicillin hydrochloride; teicoplanin; temafloxacin hydrochloride; temocillin; tetracycline; tetracycline hydrochloride; tetracycline phosphate complex; tetroxoprim; thiamphenicol; thiphencillin potassium; ticarcillincresyl sodium; ticarcillin disodium; ticarcillin monosodium; ticlatone; tiodonium chloride; tobramycin; tobramycin sulfate; tosufloxacin; trimethoprim; trimethoprim sulfate; trisulfapyrimidines; troleandomycin; trospectomycin sulfate; tyrothricin; vancomycin; vancomycin hydrochloride; virginiamycin and/or zorbamycin.

Antimicrobials can be biocompatible when the toxicity that is demonstrated is tolerable for the intended utility. Thus, for human utility, biocompatible can be acceptably toxic or non-toxic to humans or human tissues.

One of the most common methods of measuring anti-microbial effect is by the Kirby-Bauer method. The Kirby-Bauer method measures the zone of inhibition (ZOI) created when the material is placed on a bacterial lawn grown onto agar. A relatively small or no ZOI (ex. less than 1 mm) indicates either a non useful anti-microbial effect or low solubility of the active agent in the media of study, while a larger ZOI (ex. greater than 5 mm) indicates a highly useful anti-microbial effect. One procedure for a ZOI test is set out in the Examples which follow.

“Sustained release” or “sustainable basis” are used to define release of atoms, molecules, ions or clusters of an anti-microbial metal that continues over time measured in hours or days, and thus distinguishes release of such metal species from the bulk metal, which release such species at a rate and concentration which is too low to achieve an anti-microbial effect, and from highly soluble salts of anti-microbial salts such as silver nitrate, or less soluble silver salts such as silver acetate, or highly soluble antimicrobial (organic species) such as polyhexamethylene biguanide dihydrochloride (PHMB or polyhexanide), or organic species with lower solubility that include chlorhexidine diacetate, octenidine dihydrochloride, or more complex salts such as octenidine polystyrene sulfonate, polyhexamethylene biguanide polystyrene sulfonate, polyhexamethylene biguanide carboxymethyl cellulose, or octenidine carboxymethyl cellulose.

The pharmaceutical agent can be an anti-inflammatory agent. The anti-inflammatory agent can be one or more of hydrocortisone, hydroxyltriamcinolone, alphamethyldexamethasone, dexamethasone-sodium phosphate, dexamethasone; beclomethasone dipropionate, clobetasolvalerate, desonide, desoxymethasone, desoxycorticosterone acetate, dexamethasone, dichlorisone, diflorasonediacetate, diflucortolonevalerate, fluadrenolone, fluclaroloneacetonide, fludrocortisone, flu methasonepivalate, fluosinoloneacetonide, fluocinonide, flucortinebutylester, fluocortolone, fluprednidene (fluprednylidene)acetate, flurandrenolone, halcinonide, hydrocortisone acetate, hydrocortisone butyrate, methylprednisolone, triamcinoloneacetonide, cortisone, cortodoxone, flucetonide, fludrocortisone, difluorosonediacetate, fluradrenaloneacetonide, medrysone, amc, amcinafide, betamethasone and the balance of its esters, chlorprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasonedipropionate, betamethasonedipropionate, triamcinolone prostaglandin H synthetase inhibitors (Cox I or Cox II), flurbiprofen, ketorolac, suprofen, nepafenac, amfenac, indomethacin, naproxen, ibuprofen, bromfenac, ketoprofen, meclofenamate, piroxicam, sulindac, mefanamic acid, diflusinal, oxaprozin, tolmetin, fenoprofen, benoxaprofen, nabumetome, etodolac, phenylbutazone, aspirin, oxyphenbutazone, NCX-4016, HCT-1026, NCX-284, NCX-456, tenoxicam, carprofen, cyclooxygenase type II selective inhibitors, vioxx, celecoxib, P54, etodolac, L-804600, S-33516; PAF antagonists, A-137491, ABT-299, apafant, bepafant, minopafant, E-6123, BN-50727, nupafant, modipafant, PDE IV inhibitors, ariflo, torbafylline, rolipram, filaminast, piclamilast, cipamfylline, CG-1088, V-11294A, CT-2820, PD-168787, CP-293121, DWP-205297, CP-220629, SH-636, BAY-19-8004, and/or roflumilast.

The active pharmaceutical agent can be an antiproteolytic agent. The antiproteolytic agent can be one or more of amprenavir (Agenerase), fosamprenavir (Lexiva), indinavir (Crixivan), lopinavir/ritonavir (Kaletra), ritonavir (Norvir), saquinavir (Fortovase), and nelfinavir (Viracept), salts of ethylene diamine tetracetic acid, salts of polystyrene sulfonate, and sulfated oligo & polysaccharides.

Composition 14 can comprise from about 60 to about 99 (wt/wt %) silicone material; from about 1 to about 40 (wt/wt %) pharmaceutical agent. Composition 14 can be prepared before partial or complete curing of the silicone material. Composition 14 can be prepared as part of the curing of the silicone material. Composition 14 can be prepared upon partial or completion of the curing of the silicone material.

Medical device 10 may be configured as a wound dressing. Composition 14 may be an adhesive mixture bound to substrate 12. The adhesive mixture can be a tacky silicone material and at least one active pharmaceutical agent. The adhesive mixture can be conformal and/or pressure sensitive.

Substrate 12 can be substantially planar. Substrate 12 can be a flexible fabric such as one or both of knitted and non-woven fabric. Substrate 12 can be cellulosic such as one or both of cotton or wool. Substrate 12 can be a polymeric material. The polymeric material can be one or both of woven or a film. Substrate 12 can be one or more of a polyurethane, polyalkylene, polysiloxane, polyester, and/or polyamide. Substrate 12 can be a fenestrated film material.

In accordance with example embodiments, composition 14, configured as an adhesive mixture, for example, can extend around the perimeter of the substrate. Substrate 12 can define an opening extending between the periphery of the plane of the substrate. The substrate can define an opening extending from an edge of the plane inwardly to a point away from the edge. The adhesive mixture may extend around the perimeter of the opening.

Alternate compositions and/or configurations of the devices of the present disclosure, including various anti-microbial formulations of composition 14 used in the devices are set out in further detail below.

As an example, composition 14 can be formed of an tacky silicone gel such as Nusil Technologies MED 6345 (platinum curing silicone elastomer) and substrate 12 may be formed of a high surface area, porous, non-adherent material such as a woven, non-woven, or fenestrated film material, fabric such as cotton, gauze, a polymeric netting or mesh such as polyethylene, nylon, polypropylene or polyester, an elastomer such as polyurethane or block copolymer elastomers such as Kraton, or a foam such as open cell polyurethane foam. Example woven meshes may be formed from polyester, acetate, or cotton gauze. One example, hydrophilic polyurethane foam is HYPOL™, available from W. R. Grace & Co., New York, N.Y., USA.

An absorbent material for use in the devices of the disclosure such as part of composition 14 or as a separate a wound contact layer or as substrate 12 such as an upper surface of a surgical site antimicrobial frame, or around transcutaneous devices such as a super-pubic catheter where leaking urine may be absorbed and held away from the skin where otherwise it can cause irritation and maceration, is a non-woven rayon/polyester core such as SONTARA™ 8411, a 70/30 rayon/polyester blend commercially available from Dupont Canada, Mississauga, Ontario, Canada. This product is sold by National Patent Medical as an American White Cross sterile gauze pad. However, other suitable absorbent materials include woven or non-woven materials, non-woven such as Evolon® spunbond polyester being preferred given the ability to absorb up to 400% of its own weight in liquid as a consequence of the high surface area. The material may be made from fibers such as rayon, polyester, rayon/polyester, polyester/cotton, cotton and cellulosic fibers. Creped cellulose wadding, an air felt of air laid pulp fibers, cotton, gauze, and other well known absorbent materials suitable for medical dressings can be utilized. Other absorbent materials which may be used for the inner portions of the barrier dressing device include foamed materials (synthetic and biopolymers), hydrogels, absorbent polymers such as hydrocolloids to include any variety known in the wound healing art including carboxymethylcellulose (CMC), alginates, collagen, hydrophilic polyurethanes, crosslinked acrylic polymers, crosslinked polyethylene glycol, silicone gel blends that incorporate absorbent polymers such as poly(vinylpyrrolidone) (PVP), polyethylene glycol (PEG), carboxymethylcellulose (CMC), alginates, or a commercially available hydrocolloid wound dressing such as Carra Colloid (Carrington Laboratories). These absorbent materials may also be formulated to include an active pharmaceutical ingredient such as an antimicrobial agent (e.g. chlorhexidine) or a healing aid such as vitamin E and the absorbent material may be connected to the bulk dressing (silicone gel) material in at least two ways that include as an island situated upon the silicone gel or recessed into the silicone gel.

Referring to FIG. 2, device 10 can be applied to epidermal layer 22 of patient 20 for example. Layer 22 may or may not include a laceration, ulcer, or other epidermal breach. Device 10 can be applied to layer 22 contacting composition 14 with layer 22 as part of a method for dressing wound for example. Of course device 10 may be applied to layer 22 for other reasons, including but not limited to post or pre surgical, or prophylatically for example. In accordance with one method, substrate 12 can be adhered to layer 22 using composition 14.

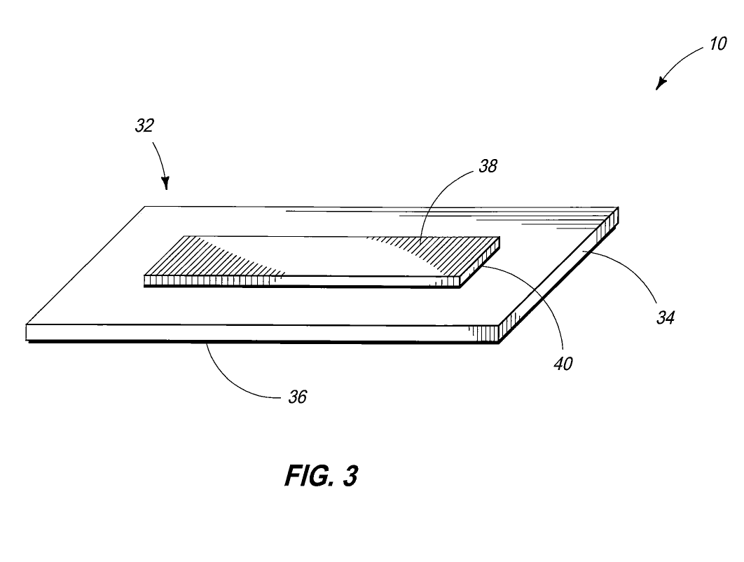

Referring to FIG. 3, a device 32 is shown configured as a surgical wound barrier dressing. Device 32 can include composition 14 configured as a silicone gel component 34 having a release liner 36 attached thereto. Device 32 can include a fluid absorbing portion bound to the substrate such as an absorbent layer 38 on the surface of component 34 opposing the surface having release liner 36 attached thereto. Absorbent layer 38 may take the form of an island above component 34 and/or may be attached to component 34 with an interfacial layer 40. The absorbent layer can be a hydrocolloid for example and may be part of composition 14 for example.

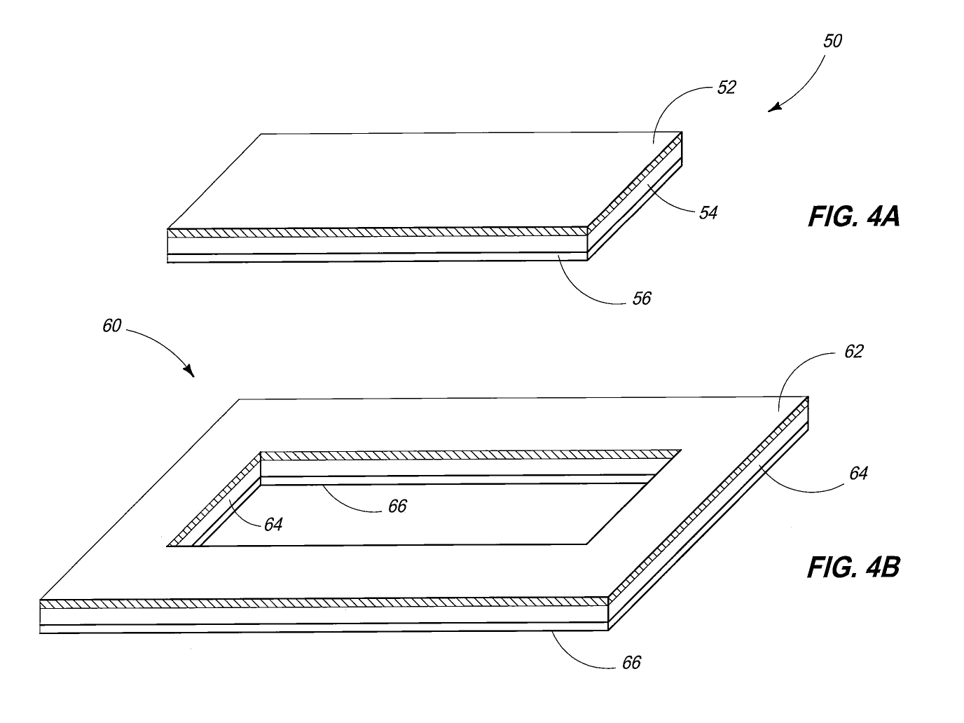

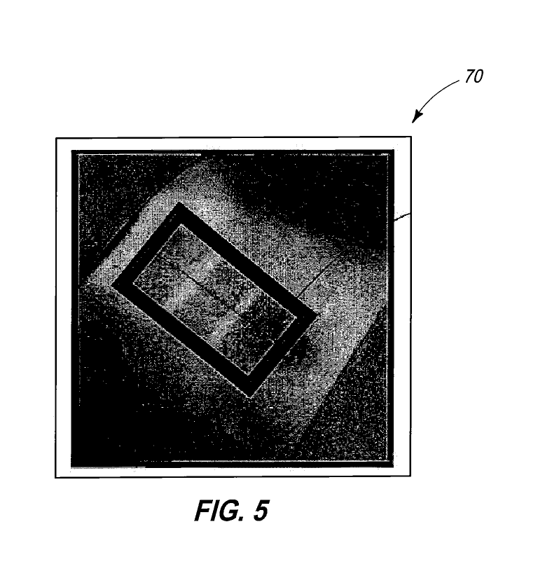

Referring to FIGS. 4A and 4B, in accordance with an alternative embodiment, devices 50 and 60 are shown than can be configured as an insert and frame. Devices 50 and 60 can be configured as an antimicrobial surgical barrier that may be implemented as part of a surgical drape or may be used alone. FIG. 5 is a representation of the surgical barrier placed on the abdomen prior to a surgical procedure. Device 70 is represented as a pictorial representation of the barrier is applied to the abdomen, for example. In accordance with example implementations, devices 50 and 60 can be constructed of composition 14 configured as a tacky silicone gel layer 54 and 64, respectively. These layers 54 and 64 may have release liners 56 and 66 on one side and substrate 52 and 62 on the opposing side. Device 50 may be used as a surgical tape to confine dressings for example, and device 60 may be used to define a barrier for a surgical procedure.

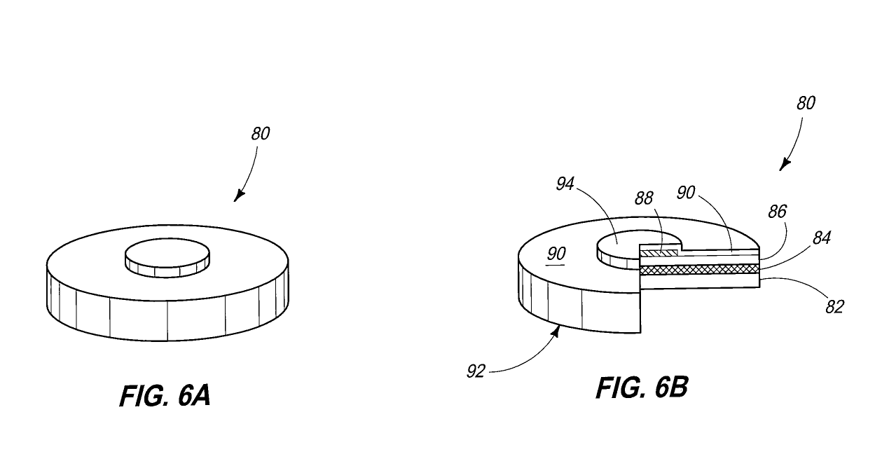

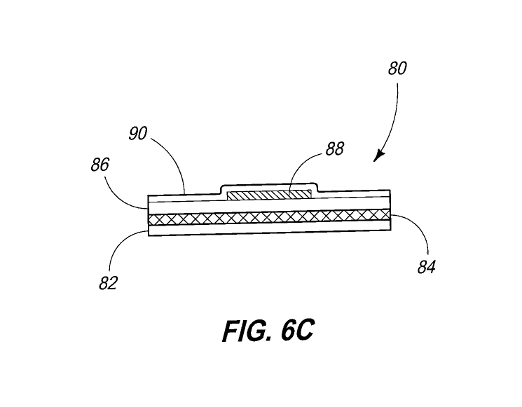

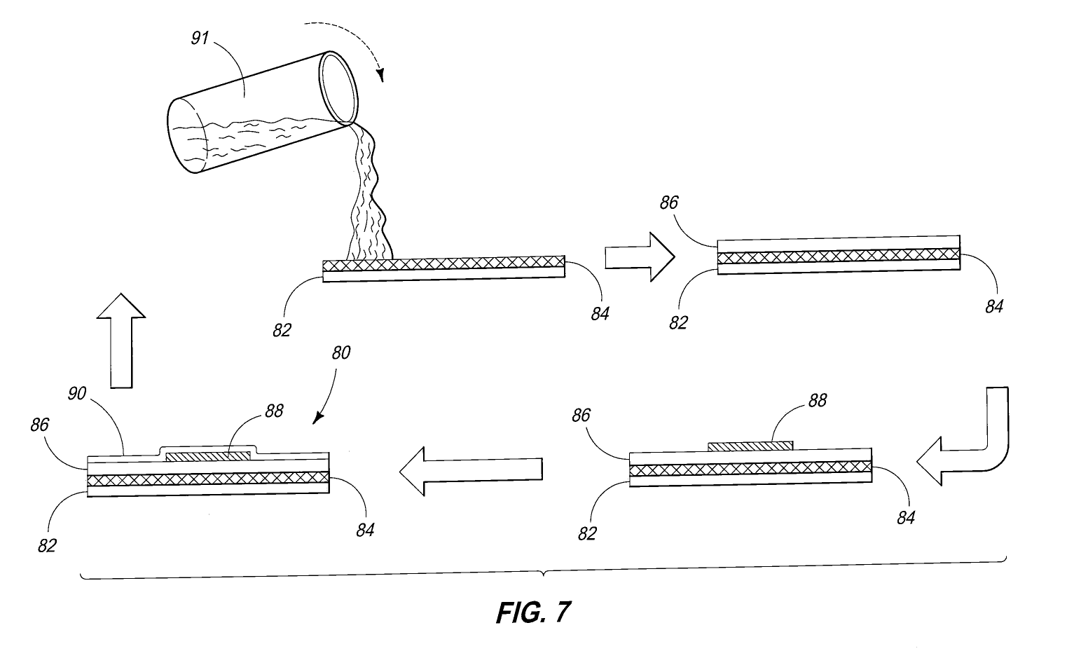

Referring to FIGS. 6-7, another embodiment of a medical device is shown as well as a process flow for producing same. Referring to FIGS. 6A-6C, device 80 is configured as a disk or puck. Device 80 can include a composition 14 configured as a silicone gel component 86 having a release liner 90 on one surface and substrate 12 configured as a fabric material 84 on the opposing surface. Device 80 can further include and an impervious flexible layer 82 on the opposing surface of fabric material 84. Device 80 can define a base 92 having an island 94 extending therefrom. Base 92 can include at least silicone gel component 86 and fabric material 84. Island 94 can extend from silicone gel component 86 and/or include an absorbent material 88. Island 94 can include release liner 90, and release liner 90 may be continuous from the one surface of silicone gel component 86 to an opposing surface of absorbent material 88. Alternatively, silicone gel component 86 can be cured as a sheet in a mold where release is facilitated easily and fabric layer 84 added to the surface. The fabric silicone composite can be turned 180 degrees and absorbent material layer 88 added. Shapes of the device can be die cut from the sheet as desired.

Referring to FIG. 7 a process flow for preparing device 80 is shown with the materials being consistent with those described in FIGS. 6A-6C. In the fabrication of such devices it can be beneficial to dispense the uncured silicone gel mixture over a composite substrate that includes a bottom layer impervious to a silicone liquid composition ready for cure and comprising a thin flexible material such as a polymer such as polyurethane and a second layer in contact with the first impervious layer comprising a high surface area material such as a polyester fabric (woven or non-woven). When cured this composition can remain flexible and the gel can be further modified to include an absorbent component and covered with a release liner, for example. The following method can also be applied to scaling up to produce island dressings or similar 5-layer configurations.

In accordance with a specific example, a platinum catalyzed 2-component system can be utilized such as Med 6345 (Nusil, of Carpenteria Calif.), comprising parts A and B which are generally combined in equal proportions (50:50 w/w) and a known quantity of the finely divided antimicrobial agent is added and the mixture combined with the aid of a spatula or by using an automated mixing apparatus such as a SpeedMixer. In practice, the antimicrobial agent may be combined with one of the constituents (part A or B) and mixed to a fine consistency before adding the second part or as a kit for storage and use at a later time. The material is cast onto a release liner, such as a polyethylene or a fluoropolymer for example and into a mold and the silicone mixture gently leveled by the use of a spatula. The barrier dressing formulation is cast onto a release liner within a curing “tool” (mold) and the tool is placed in a convection oven set at 80° C. and the formulation cured for a period of 2-3 hours to yield a tacky gel substrate with a release liner affixed to one side. The protective layer is subsequently pressed onto the available silicone gel surface and the protective layer is retained by contact adhesion. By using a slight excess of part B, such as a 55:45 w/w of part B to part A, the gel may be formulated to have increased firmness.

In yet another example of a silicone material that can be used, a proprietary UV-curing silicone gel (Momentive Performance Materials, Albany, N.Y. 12211). Part A and part B (catalyst) are mixed thoroughly with the desired quantity of antimicrobial agent and the formulation cast onto a release liner contained by a curing “tool” (mold) and the tool placed onto a Dymax conveyer system with a Fusion UV system employing an Iron D bulb. The silicone was readily cured following 2 passes at a 2 ft/minute exposure rate.

The devices may be sterilized using gamma or electron beam radiation is preferred for sterilizing such barrier dressings, as discussed in U.S. Pat. No. 5,454,886, the entirety of which is incorporated by reference herein. The sterilized dressings should be sealed in packaging which excludes light penetration to avoid oxidation of any of the anti-microbial additives. Polyester, or metalized polymer and heat sealable pouches are preferred. The shelf life of anti-microbial dressings sealed in such a fashion is at least one year.

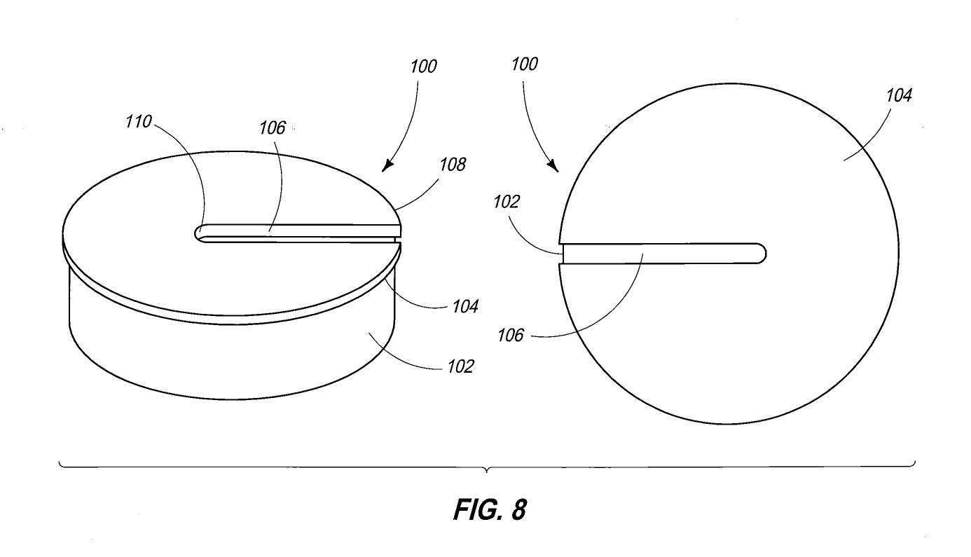

Referring to FIGS. 8-15 other embodiments of medical devices are depicted. The medical devices may be single or multi-component medical devices and they may be utilized as dressings for transcutaneous devices. Referring to FIG. 8, device 100 can include at least two layers of medical dressing materials laminated together by contact adhesion. Device 100 includes a two-layer silicone gel construct dressing in accordance with the disclosure can include a first layer 102, which will be skin facing in use, a second layer 104 which preferably forms an protective layer. The layers 102 and 104 are shown to be laminated together by contact adhesion onto one surface of 10. In accordance with the disclosure, layer 102 can be considered composition 14 and layer 104 can be considered substrate 12. Layer 104 can include an opening 106 extending from a perimeter of layer 104 to a portion of layer 104. This opening can be configured in various shapes such as a circular opening to receive a transcutaneous device, for example.

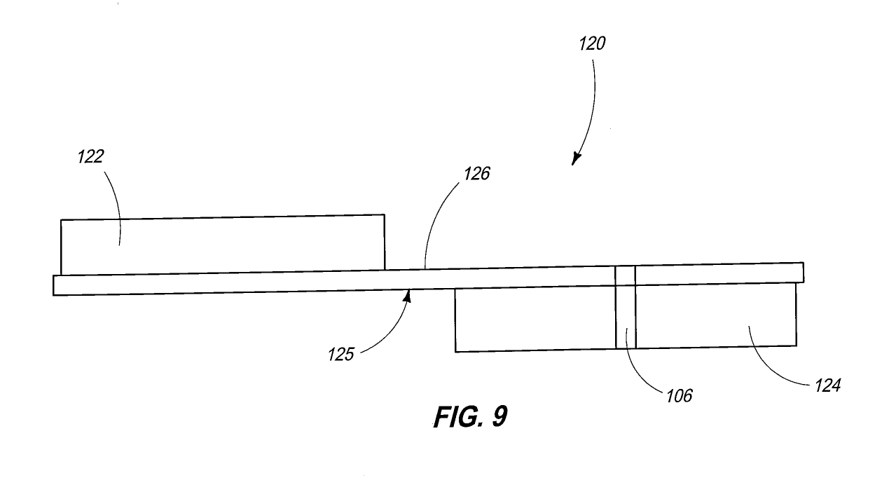

Referring to FIGS. 9-11, medical device 120 is shown that includes a base 124 having an opening 106 configured to receive a transcutaneous medical device. Base 124 can include composition 14 configured as a tacky substance such as a tacky silicone gel and composition 14 can be associated with one surface configured to engage a portion of epidermis of a patient. Device 120 can also include a cover 122 associated with base 124 and configured to mate with another surface opposing the one surface of base 124.

Cover 122 and base 124 can be associated via a common substrate 12 such as fabric 126. As shown, cover 122 and base 124 can be situated 180 degrees apart on opposite sides of the fabric 126. In accordance with example configurations, In FIG. 2, cover and base can be mated by folding pliable substrate 126 at hinge 125. As an example this can provide an adhesion boundary between the cover and base and the common central piece of fabric 126.

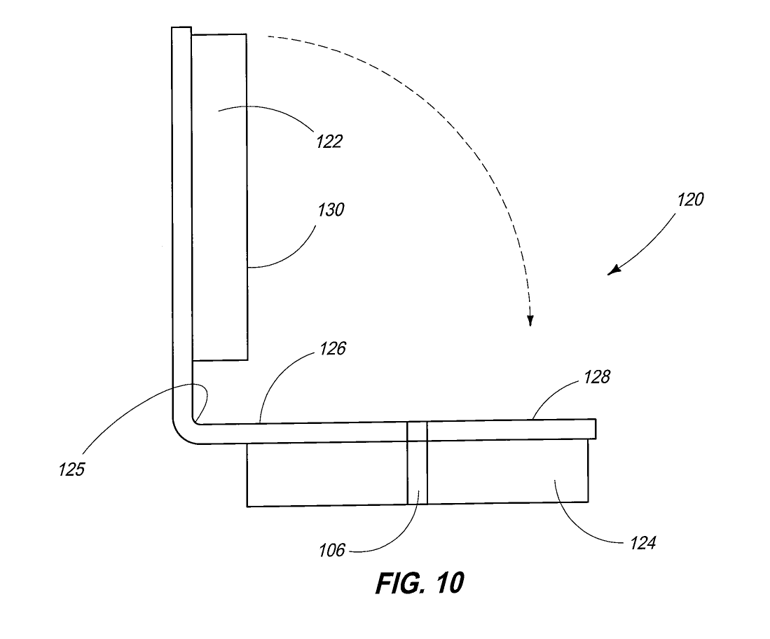

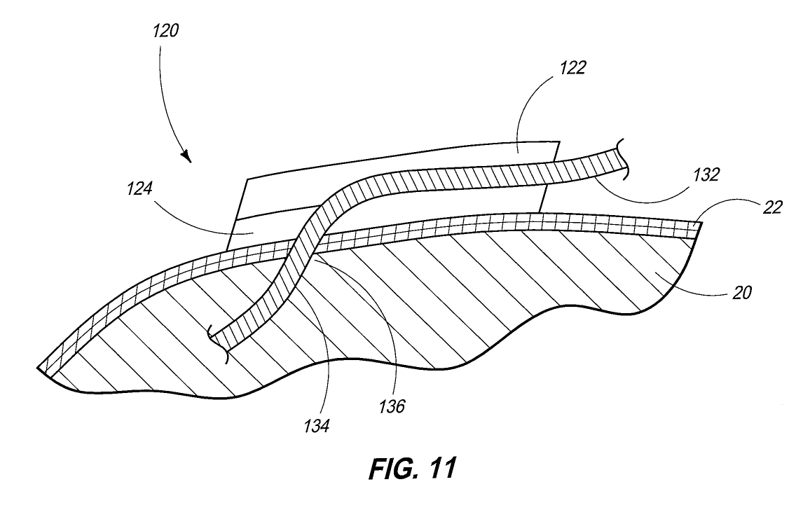

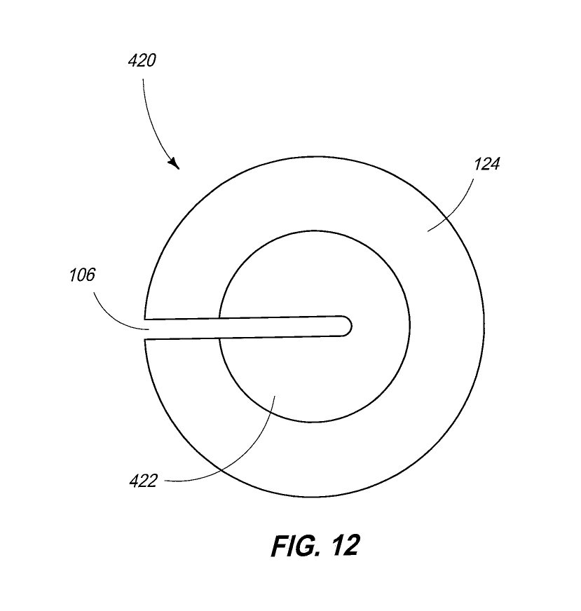

Referring to FIG. 10, cover 122 and base 124 are shown at 90° from each other and intimating how the adhesion of cover 122 to substrate 126 can be accomplished. Referring to FIG. 11 depicts device 120 securing transcutaneous device 132 with the cover 122 adhering to substrate 126 and securing device 132 through epidermal layer 22. Base 124 of device 120 can further define a trough extending from the perimeter of the base to the opening with the trough configured to receive at least a portion of the transcutaneous device. FIG. 12 details device 120 from a top down perspective detailing slit 106 and inner absorbent material 422, for example.

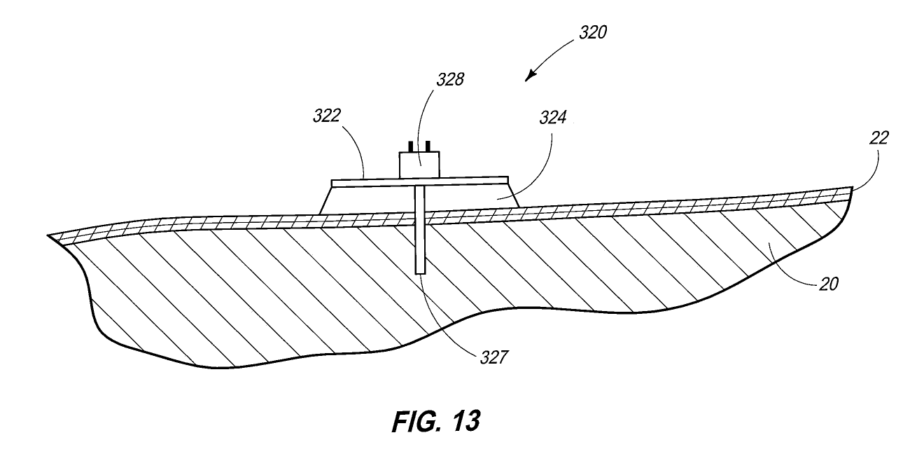

Referring to FIG. 13 an embodiment of a medical device 320 is shown securing a subcutaneous glucose sensor 327 piercing the dermis 22 with its electronic connector 328 sitting external to body 20.

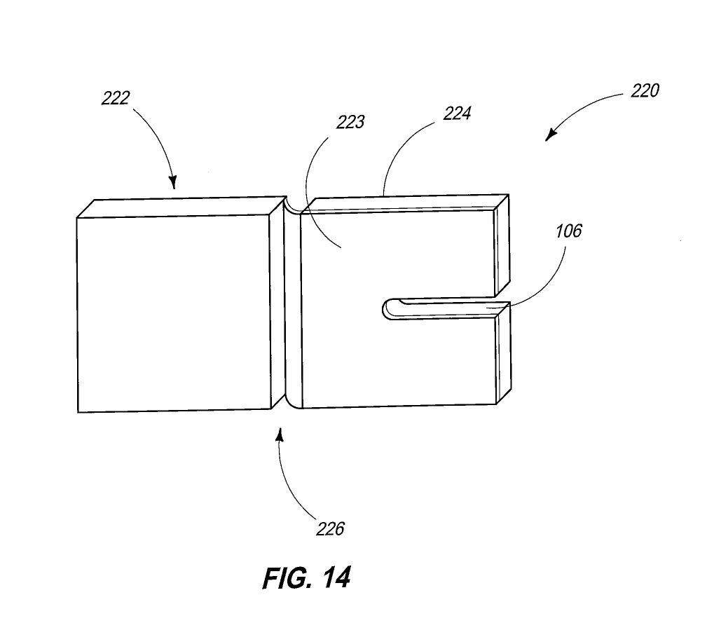

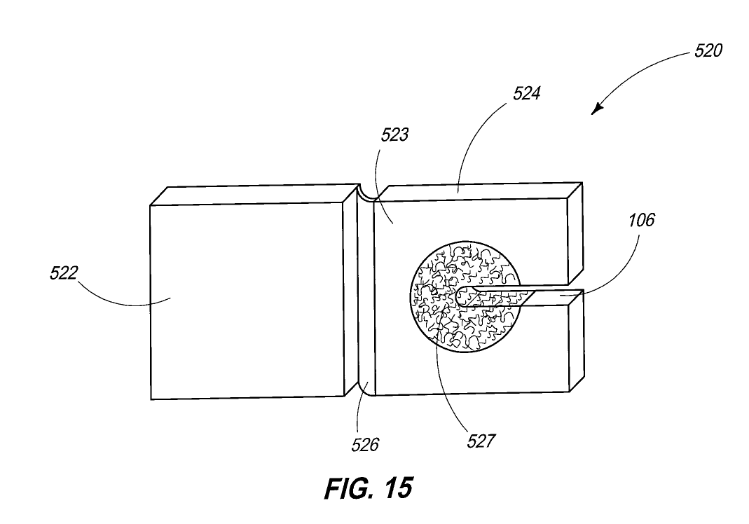

FIGS. 14 and 15 detail alternative cover/base device 222 as a three-layer device 220, to include a inner absorbent material 527 surrounding tacky silicone barrier 224 and protective top layer material 524.

In accordance with this disclosure, the cover and base may be may be formulated separately to include different additives. For example, the base may include an antimicrobial agent and the cover may be free of any additive. In another embodiment, the base and cover can both be formulated to include an antimicrobial agent. In yet another embodiment, the base may include an organic-based antimicrobial agent and the cover can include an antimicrobial metal salt such as silver acetate thus alleviating contact of the skin by a silver salt containing polymer, which could lead to skin discoloration. The cover and base may be of equivalent thicknesses or different thicknesses and may range from approximately 3 mm to approximately 15 mm, for example.

Also in accordance with this disclosure, the substrate may be a polymer material that includes polyurethanes, polyalkylenes, polysiloxanes, for example in sheet or film or foam form or it may be a polymer material in fabric form such as a woven, non-woven, or knitted fabric that includes polyesters, polyamides, and cellulosics that include natural fibers such as cotton, wool or the like. Additionally, the substrate may be coated with the previous or other materials alone or in combination, such polyurethane, for example. Furthermore, the substrate may be designed to wick fluid from the center of the device by controlling the thickness and surface area of the material; for example, using a non-woven material such as a polyester. Substrate thicknesses may span a range of approximately 0.6 mm to approximately 5 mm, for example.

The medical devices as dressings can be sized to cover a significant portion of the device 132 that protrudes from the skin 22, and not just the immediate skin area surrounding the penetration site. This may aid in limiting infection, since bacterial migration along the skin to the device 132 are minimized. A minimum dressing size will preferably provide at least 5 cm of protruding device 132 coverage, and more preferably 15 cm coverage. Depending on the size of the transcutaneous medical device, the termination point of the slit 106, may include additional cuts, preferably a cross-cut, or a penetrating hole (not shown), to allow the dressing barrier to fit around the device while still maintaining close contact with both the skin and protruding section of the medical device.

FIG. 12 shows device 420 from a top-down view with protective substrate 124 and slit 106. Device 420 further includes absorbent material 422 above a portion of substrate 124.

FIG. 13 demonstrates placement of device 320 over epidermis 22 of patient 20. Substrate 322 can be supported by composition 324, for example, a protective layer over tacky silicone gel, with or without a pharmaceutical agent. Device 320 can secure a biosensor with connector 328 above substrate 322 and transcutaneous lead 327 below epidermis 22. Composition 324 can adhere to dermis 22 securing the biosensor in place. The biosensor can be an enzymatic sensor based upon glucose oxidase for example. Generally, these glucose sensors evaluate glucose by electrochemically measuring hydrogen peroxide resulting from the enzymatic digestion of glucose by glucose oxidase.

FIGS. 14 and 15 represent alternative embodiments of cover/base medical device configurations. Referring first to FIG. 14, medical device 220 can include base 224 with opening 106. Base 224 can be associated with cover 222 via a hinge portion 226, for example. Base 224 can include a substrate 223 which may form part of hinge portion 226. FIG. 15 represents another device embodiment, device 520, that can include base 524 and cover 522 associated therewith. Base 524 can include substrate 523 thereover, which may extend and attach to cover 522 via hinge 526. Above substrate can be absorbent material 527 which may also extend into opening 106.

As described herein, the disclosure provides devices and methods for its use with a transcutaneous medical device, such as an intravascular catheter, which punctures the skin of a patient and which has a portion of the medical device protruding from the skin or to dress a wound that may be surgically created or created by trauma. These transcutaneous devices are prone to infection as a consequence of easy access of bacteria to the open wound. As described, the devices of the present disclosure can be formed from a flexible, adherent silicone gel having upper and lower surfaces, with the lower surface being the skin facing surface in use and the top adherent surface protected by a substrate such as a flexible woven, knitted, or non-woven fabric. The device may have an opening formed therein extending from one edge inwardly to a termination point within the confines of the device. Current dressings for protection against infection of transcutaneous devices generally utilize a secondary transparent film (adhesive) dressing, such as with BIOPATCH®, in order to secure the device around the transcutaneous device. It has been recognized that these secondary dressings can irritate skin (allergic response) and damage epithelium when removed thus leading to further tissue irritation. The compositions of the devices of the present disclosure can be more gentle to skin upon removal and less likely to lead to an allergic response.

Specific embodiments of these devices may or may not include one or more of a pharmaceutical agent or other functional agents such as a protease inhibitor incorporated into the silicone gel for placement adhered above the wound, without the use of additional adhesives at the upper and lower surfaces of the device. As an example use, the device can be placed next to the skin, the opening allowing the base to surround the puncture site and at least a portion of the transcutaneous device protruding from the skin such that the lower surface of the base of the device is in contact with the skin while the upper surface of the base of the device is also in contact with a portion of the transcutaneous device protruding from the skin. A cover may be utilized and joined to the base and folded onto the top portion of the base such that the lower surface of the cover is in contact with the upper surface of the base and at least a portion of the transcutaneous device so as to secure the transcutaneous device and provide another layer of antimicrobial protection. In accordance with aspects of the disclosure, the portion of the transcutaneous device protruding from the skin can be exposed to composition 14 such as antimicrobial (silicone) material of the base. When affixing the cover to the base, a greater surface area of the transcutaneous device may also be exposed to composition 14 as part of the silicone gel of the cover secures the transcutaneous device to the substrate of the base.

The embodiments herein describe devices and/or methods for preventing infection, protein drug degradation, prolonging the use of transcutaneous devices, and/or protecting wounds, including surgical wounds from external or bacterial insult is disclosed. Embodiments of the disclosed medical device are conformal, and can include an adhesive silicone barrier dressing surrounding a device that breaches the skin of a patient undergoing diagnostic, drug, or nutritional therapy, or encompassing a surgically created wound or placing just prior to the creation of a surgical wound. The conformal pressure sensitive barrier may be compounded to include one or more of a tissue preserving agent, a drug preserving agent, an antibiotic agent, an antibacterial agent, a pain suppressing agent, or a tissue healing agent and/or the device may be constructed to include an absorbent layer. Devices that may benefit from the conformal barrier device include, but are not limited to, biosensors, infusion devices, venous access devices, feeding tubes, wound drainage tubes, orthopedic pins, stomas, surgical drapes, catheters, and the like. Surgical wounds that may benefit from a conformal antimicrobial barrier device that has the ability to absorb some wound exudate include those surgical incisions created in any variety of locations on the human body. The conformable barrier device can be flexible and adhesive thus allowing the device to follow difficult contours such as from the top of the foot onto the shin (90 degree angle) or under the arm/armpit where a dressing may actually be required to fold back on itself (180 degree angle). The soft-and-supple nature of the device can allow these kinds of wound cover to be worn in comfort and to act as a barrier to the ingress of pathogenic microorganisms.

Embodiments of the disclosure a device such as a dressing barrier device that can include a tacky silicone gel construct formulated with an active pharmaceutical agent that has been finely divided and put through a sieve in order to ensure that the size of the particles is below a certain size. The device can be placed to overlie and surround an open wound supporting a transcutaneous device in order to minimize bacterial infiltration, prevent tissue loss, and provide stability and protection to active pharmaceutical agents susceptible to degradation resulting from inflammation. In addition, the device may be placed to overlie and surround a (closed) surgical wound in order to minimize bacterial infiltration, prevent external insult, provide a moist and antibacterial environment to the healing wound, and/or provide a healing aid (e.g. skin moisturizer) at the site of incision (staples or sutures).

In a another embodiment, composition 14 of the device can include the active pharmaceutical agent, the silicone gel, and in the form of a concentric part, a hydrocolloid or other water absorbing material in order to aid with fluid absorption around the wound site. In another embodiment, the device includes both a base and cover; the cover can be extended to form two distinct tacky silicone gel constructs, one of the cover and one of the base, bound to opposite sides of substrate 12 such as a protective layer and preferably with a non-overlapping boundary between the two constructs. In this arrangement, the base can be adhered to the skin surface surrounding the transcutaneous implant; the transcutaneous device is surrounded by the base with the transcutaneous device resting perpendicular to an opening in the base. The cover can then be folded onto the base to envelop the transcutaneous device and fix it in place while adhering to the upper protective layer of the base. The upper protective layer can be a high surface area material such as a woven polyester fabric, for example.

Embodiments of the disclosed devices have application to transcutaneous medical devices such as those listed above, made from a wide variety of materials, for example metals, including steel, titanium and aluminum and their alloys, latex, nylon, silicone, polyester, polyurethane, and other plastics and rubbers. Such transcutaneous devices are generally made of bioinert or biocompatible materials. The transcutaneous device may take a variety of shapes including rod or tube shapes, hollow or solid, and may be rigid or flexible, factors dictated by its intended utility. One example transcutaneous device includes an infusion set used with an infusion pump as part of intensive drug therapy. The purpose of an infusion set is to deliver a drug under the skin or into a central venous access point. It is a complete tubing system to connect an infusion pump to the infusion system generally including a catheter, subcutaneous cannula, adhesive mount, quick-disconnect, and a pump cartridge connector.

Devices of the disclosure may have application to surgically created wounds that may be sutured closed or remain open with the device creating a barrier that surrounds the wound site and the devices of the disclosure may, or may not provide an absorbent material that sits over the open or closed wound. In addition, the absorbent material may be formulated to include an antimicrobial agent or any other number of agents that may aid the healing of the wound.

Use of the devices of the disclosure as barrier dressings such as with transcutaneous devices such as flexible catheters, for example, can include a release liner fitted onto the contact layer of the barrier dressing which is subsequently removed prior to the device being placed on the skin and around the transcutaneous device (e.g. catheter) by passing or placing the catheter through the opening in the base contact layer. In an alternative procedure, the barrier dressing is first placed around the transcutaneous device and the release liner (which has been slit in a separate operation) is removed one-half at a time. Prior to removal of the release liner, the dressing can be rotated, oriented, and situated, if needed, to ensure that the opening is roughly perpendicular to the long axis of the catheter, thus ensuring that the portion of the catheter protruding from the skin is contacted by the upper surface of the base. Once the base is secure, the release liner of the cover is removed and the cover folded over the base such that the lower surface of the cover is in contact with the portion of the catheter protruding from the skin.

If the transcutaneous device is rigid, such as a temporary orthopedic pin, the base can be put in place as set out above, and the cover can then be folded and secured around the portion of the pin protruding from the skin, in a tent-like manner or in another embodiment the cover is slit and each portion is placed independently in an overlapping fashion, since the pin generally protrudes at an angle normal to the skin surface. One or more slits, holes, or openings may be provided in the cover in order to accommodate any type of transcutaneous device. Furthermore, the cover may be configured to include a secondary adhesive layer, such as a clear adhesive medical tape such as a clear acrylic. The clear adhesive layer may be provided in place of the silicone gel cover or in addition to the silicone gel cover. The clear adhesive can be affixed to the fabric layer and provided with a release liner for rapid placement over the transcutaneous device.

In vitro experiments involving embodiments of the present disclosure, formulated to include antimicrobial active pharmaceutical agents manufactured as described above and as described in greater detail in the examples have been shown to control bacterial growth.

### EXAMPLES OF FORMULATIONS FORMED INTO BARRIER DRESSING CONFIGURATIONS

### Example 1

### Nusil Med 6345 Formulated with Chlorhexidine Diacetate (CHXDA

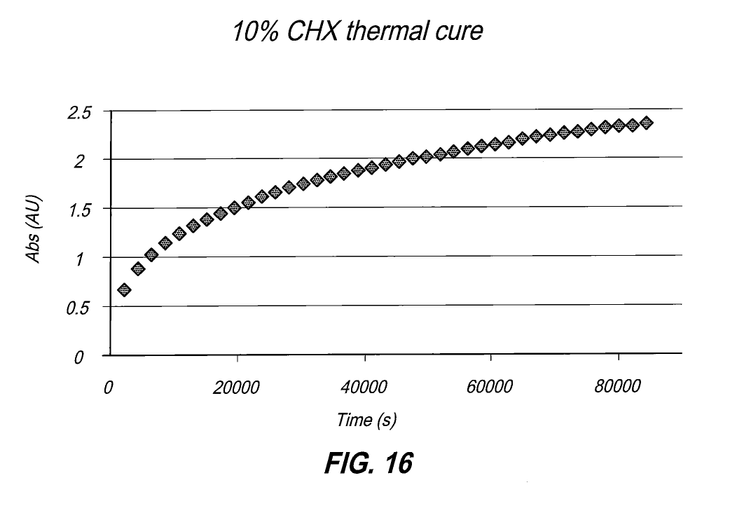

10% CHXDA
   13.995 g Part A, 13.996 g Part B, 3.11 g CHXDA combined and stirred by hand to homogeneity.
   The release profile for chlorhexidine is shown in the following graphical representation in FIG. 16.

### Example 2

### Nusil Med 6345 Formulated with Polyhexanide Hydrochloride (PHMB)

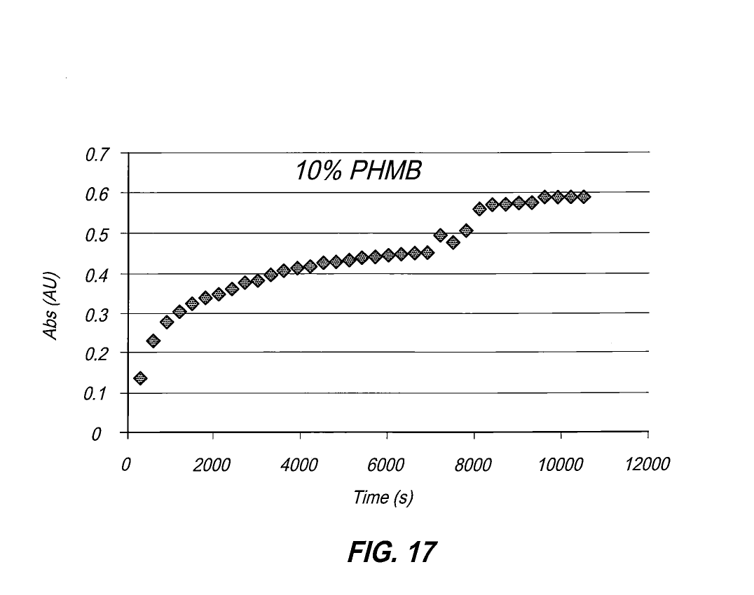

10% PHMB
   14.04 g Part A, 14.02 g Part B, 3.12 g PHMB
   The release profile for polyhexanide is shown in the following graphical representation in FIG. 17.

### Example 3

### Nusil Med 6345 Formulated with Octenidine Dihydrochloride

15% Oct.
   14.03 g Part A, 13.99 g Part B, 4.94 g octenidine

### Example 4

### Nusil Med 6345 Formulated with Silver Acetate

1% Ag acetate
   13.47 g Part A, 16.47 g Part B, 3.04 g silver acetate
   45:55 A:B

### Example 5

### Nusil Med 6345 Formulated with Polyvinylpyrrolidone (PVP) and Polyhexanide Hydrochloride (PHMB)

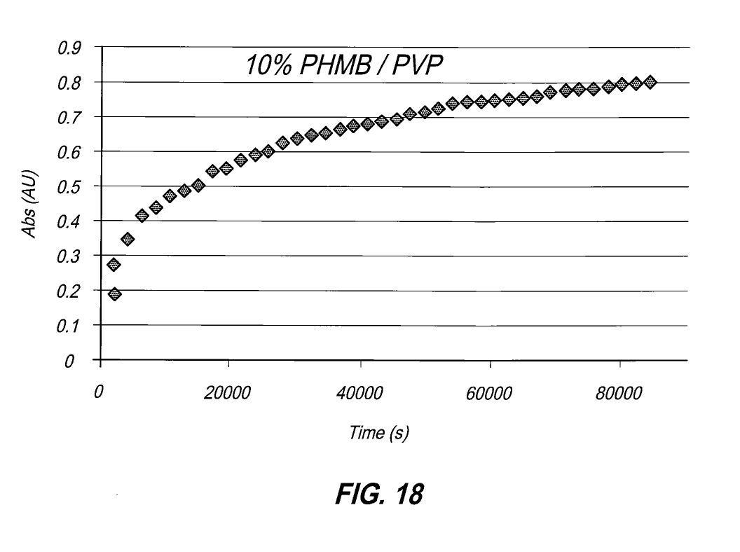

10% total solids (1:1 PHMB:PVP)
   14.02 g Part A, 14.02 g Part B, 3.12 g PHMB/PVP
   The release profile for polyhexanide is shown in the following graphical representation in FIG. 18.

### Example 6

### Nusil Med 6345 Formulated with Carboxymethylcellulose and Chlorhexidine Diacetate

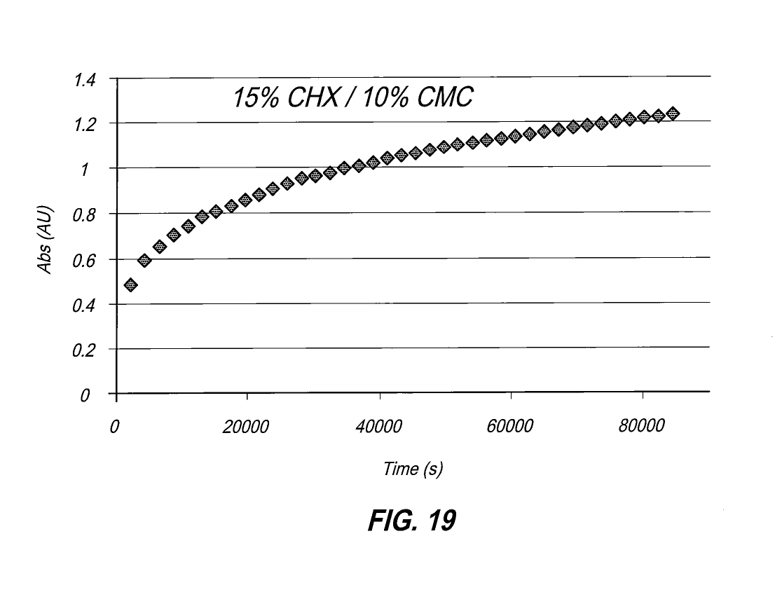

15% CHX-DA
   14.00 g part A, 14.02 g part B, 7.15 g CHX-DA, 1.75 g CMC
   The release profile for polyhexanide is shown in the following graphical representation in FIG. 19.

### Example 7

### Nusil Med 6345 Formulated with Sodium Polystyrene Sulfonate (Crosslinked, Amberlite IRP69)

20% IRP69 (available from Rohman and Haas)
13.2 g part A, 13.2 g part B, 6.6 g IRP69

### Example 8

### NusilMed 6345 Formulated with Silver Polystyrene Sulfonate (Crosslinked, Amberlite IRP69, Silver Modified)

20% IRP69
13.2 g part A, 13.2 g part B, 6.6 g IRP69

In compliance with the statute, embodiments of the invention have been described in language more or less specific as to structural and methodical features. It is to be understood, however, that the entire invention is not limited to the specific features and/or embodiments shown and/or described, since the disclosed embodiments comprise forms of putting the invention into effect.

1-9. (canceled)

10. A wound dressing comprising:
a substrate; and
an adhesive mixture bound to the substrate, the adhesive mixture comprising tacky silicone material and at least one active pharmaceutical agent.

11. The wound dressing of claim 10 wherein the substrate comprises a flexible fabric.

12. (canceled)

13. The wound dressing of claim 10 wherein the substrate is substantially planar.

14. The wound dressing of claim 13 wherein the substrate defines an opening extending from the periphery of the plane to the substrate to a portion between the periphery of the plane of the substrate.

15-16. (canceled)

17. The wound dressing of claim 13 wherein the substrate defines an opening extending from an edge of the plane inwardly to a point away from the edge.

18-19. (canceled)

20. The wound dressing of claim 10 further comprising a hydrocolloid associated with the substrate.

21. (canceled)

22. The wound dressing of claim 10 wherein substrate is a polymeric material.

23. (canceled)

24. The wound dressing of claim 10 further comprising a fluid absorbing portion bound to the substrate.

25. (canceled)

26. The wound dressing of claim 10 wherein the substrate defines a tape configured to affix medical devices to patients.

27. The wound dressing of claim 10 wherein the substrate is one or more of a polyurethane, polyalkylene, polysiloxane, polyester, and/or polyamide.

28. The wound dressing of claim 10 wherein the substrate is a cellulosic.

29. (canceled)

30. The wound dressing of claim 10 wherein the substrate comprises a fenestrated film material.

31-57. (canceled)
